(19) United States
(12) Patent Application Publication
(10) Pub. No.: US 2013/0039981 A1
(43) Pub. Date: Feb. 14, 2013

(54) Quick Dissolving, Long Acting Zinc Therapeutic Formulations

(71) Applicant: Subraman Rao Cherurkuri, Vienna, VA (US)

(72) Inventor: Subraman Rao Cherurkuri, Vienna, VA (US)

(21) Appl. No.: 13/557,432

(22) Filed: Oct. 25, 2012

Related U.S. Application Data
(60) Provisional application No. 61/512,545, filed on Jul. 28, 2011.

(51) Int. Cl. A61K 9/20; A61K 31/191; A61K 31/19; A61K 33/34; A61K 33/30; A61P 31/04; A61P 11/14; A61P 11/10; A61P 31/12; A61P 29/00; A61P 25/00; A61P 31/10; A61P 35/00; A61P 37/08; A61P 11/02; A61P 5/00; A61P 3/02; A61P 1/10; A61P 1/04; A61P 3/06; A61P 9/06; A61P 3/00; A61P 25/22; A61P 9/08; A61P 31/00; A61P 25/26; A61P 1/00; A61P 25/20; A61P 1/12; A61P 9/10; A61P 9/12; A61P 25/06; A61P 9/00; A61P 25/18; A61P 7/02; A61P 1/08; A61P 21/00; A61P 1/06; A61P 15/00; A61P 3/04; A61P 21/06; A61P 11/06; A61K 9/00

(52) U.S. Cl. 424/464; 424/484; 424/486; 424/485; 424/488; 514/494; 424/637; 424/643

(57) ABSTRACT

The present invention comprises a quick dissolving, long acting zinc therapeutic cold formulation containing high levels of an active compound encapsulated within bioadhesive/muco-adhesive polymers as a controlled release oral drug delivery system. The composition allows for increased residence time for enhanced prophylactic and therapeutic efficacy within the mouth and oral cavity. This allows for a reduction in the number of doses necessary to achieve therapeutic relief which will result in increased patience compliance.

### FIELD OF THE INVENTION

The present invention relates generally to controlled release, orally administratable pharmaceutical compositions for the treatment of colds, the flu inflammation, pain other bodily ailments in humans. More specifically, the present invention relates to a rapid-melt composition for delivery of a prophylactic and therapeutic pharmaceutical active to a mammal as well as methods for making and using the same. Preferably, the prophylactic or therapeutic active is a mineral salt.

### BACKGROUND OF THE INVENTION

Controlled release formulations of pharmaceutical agents is an extremely large market in the pharmaceutical and medical fields. A number of types of controlled release dosage forms are known, including matrix tablet systems incorporating active ingredients, fillers and various types of excipients. The very different properties of numerous different types of pharmaceutically active ingredients has necessitated the development of a number of different drug delivery systems utilizing polymer technology in order to provide an appropriate release of a particular medicament after oral ingestion by a patient. Tablet oral dosage forms compositions offer many advantages, including ease of product handling, chemical and physical stability, portability (in particular, allowing ready availability to the consumer when needed), aesthetic acceptability and dosage precision, i.e., ensuring consistent and accurate dosages of the pharmaceutical active.

However, liquid formulations may offer advantages in the treatment of certain disorders, such as disorders of the upper gastrointestinal tract, wherein delivery of an active material dissolved or dispersed in a liquid ensures rapid and complete delivery to the afflicted area. In an effort to obtain the therapeutic advantages associated with liquid formulations as well as the broad advantages associated with solids, many chewable tablet formulations have been developed. Solid dose delivery of bioactive materials to mucosal, dermal, ocular, subcutaneous, intra-dermal and pulmonary tissues offers several advantages over previous methods such as topical applications of liquids, transdermal administration via so-called “patches” and hypodermic injection. In the case of injection, solid dose delivery can reduce the risk of infection by eliminating the use of needles and syringes, provide for more accurate dosing than multi-dose vials, and minimize or eliminate the discomfort which often attends hypodermic injection.

One important factor in formulating oral dosage forms such as chewable tablets, beads, powders, granules, is palatability and mouth feel, especially in tablets that include pharmaceutical dosages. Many pharmaceutical and confectionery tablets are designed to be chewed either to provide proper flavor or to increase the surface area of a particular drug to permit rapid activity in the digestive tract or circulatory systems. However, many pharmaceutical ingredients usually have both an unpleasant mouth feel and unpalatable taste due to chalkiness, grittiness, dryness and astringent properties of these materials. Accordingly, the practical value of these materials is substantially diminished since patients finding them objectionable may fail to take them as prescribed. A number of formulations have been investigated to ease the mouth feel and palatability of such compositions.

The unique novel combination of the components of the present invention allows for fast melting of the composition when placed in the mouth of a user. By pressing the composition between the tongue and cheek of the user, the saliva of the user provides hydration to the composition and allows the composition to melt without any chewing. A unique feature of the present inventive compositions is that the composition becomes a liquid upon the application of pressure. The compositions rapidly melt upon the application of pressure by the tongue of the patient, thus forming a liquid carrier for the active ingredients contained therein. The liquid helps provide the unique characteristics of the present invention.

The liquefaction of the inventive compositions can be achieved through the application of pressure by the tongue of the patient, as described above. Optionally, the liquefaction may be attained by the patient chewing the compositions. A slight amount of chewing will enhance the liquefaction of the compositions. A further way for the composition to be liquefied is by the patient sucking on the rapid-melt, compositions of the inventive subject matter. The rapid-melt technology of the present inventive subject matter has multiple applications which are ideal for the unique properties of the compositions. One such application is the delivery of active ingredients to a mammal in need thereof.

The mucosal route of drug delivery, in particular the sublingual or nasal mucosal routes, offer a useful alternative to parenteral delivery where a rapid therapeutic effect is desired. Sublingual use of the commercially available oral tablet dosage forms of alprazolam offers no significant benefit over conventional oral-gastric administration in terms of speed of onset [see J. M. Scavone et. al., J. Clin. Psychopharmacol., 1987, 7, 332 335]. Formulation of many drugs in a manner which permits rapid uptake from the sublingual mucosa allows for the quick relief of many cold and flu symptoms.

However, the low permeability of the membranes that line the oral and nasal cavities result in a low flux of the drug. There is therefore a need to enhance drug solubility and penetration to improve bioavailability following oral mucosal drug delivery. There is also a need to increase the residence time of the drug in the oral cavity to enhance the bioavailabilty of the active and thereby improve the therapeutic efficacy of the active so administered. Whereas there are many methods known in the art to deliver drugs to the oral and nasal mucosae which include buccal and sublingual tablets or lozenges, adhesive patches, gels, solutions or sprays (powder, liquid or aerosol) for the oral cavity and liquid or aerosol solutions or sprays for the nasal cavity, there is still a need for significant Improvement.

The absorption of drugs from the carrier delivery system by the mucosal membranes may be enhanced by (i) increasing drug solubility, (ii) modifying the pH of the system to favor the un-ionized form of the drug, (iii) adding muco-adhesive agents to improve contact between the delivery system and the membrane and, (iv) incorporating so-called penetration enhancers.

U.S. Pat. No. 6,024,981 to Khankari et. al., discloses a rapidly dissolving robust dosage form directed to a hard tablet that can be packaged, stored and processed in bulk. The solid tablet dissolves in the mouth of a patient with a minimum of grit. The tablet contains an active ingredient mixed into a matrix of a non-direct compression filler and a relatively high lubricant content.

U.S. Pat. No. 5,989,583 to Amselem, discloses a dry solid lipid composition suitable as an oral dosage form. The composition contains a lipophilic substance, at least one fat which is a solid at about 25.degree. C. and at least one phospholipid present in an amount of about 2 to 40% by weight of the composition. However, the resultant product is a dry solid lipid composition.

U.K. Patent Application. GB 2,195,892,892 to Amselem discloses pharmaceutical chewable tablets with improved palatability. The lipid-containing tablets include a lipid material having a melting point from about 26° C. to about 37° C., a particulate dispersant material, an emulsifier and a safe and effective amount of a pharmaceutically active material. The tablets of the lipid composition exhibit improved palatability, and effective dispersion in the mouth and stomach. pharmaceutical chewable tablets with improved palatability. composition exhibit improved palatability, and effective dispersion in the mouth and stomach.

U.S. Pat. No. 5,837,285 to Nakamichi et. al., discloses fast soluble tablets that can be produced by a simple method. The tablet base is a sugar alcohol. The mixture of the sugar alcohol and a drug is subjected to compressive shaping prior to drying in the process. The dry solid tablet can be produced by modification of conventional tabletting technology and possesses physico-chemical stability.

U.S. Pat. No. 5,753,255 to Chavkin et. al. discloses a chewable medicinal tablet. The tablet contains about 30 to about 95% by weight of a capric triglyceride and a medicinally active ingredient up to 60% by weight. If the medicinally active ingredient is less than about 30% by weight, then the composition also contains up to 10% by weight of a member of the group consisting of glyceryl monostearate, a mixture of glyceryl monostearate and glyceryl monopalmitate, and a mixture of glyceryl monostearate and glyceryl distearate.

U.S. Pat. No. 5,320,848 to Geyer et. al. discloses a non-aqueous chewable composition for oral delivery of unpalatable drugs. The drug is intimately dispersed or dissolved in a pharmaceutically-acceptable lipid that is solid at room temperatures. The lipid material desirably readily melts with the application of mild temperatures, i.e. about 55 to 95 C.

U.S. Pat. No. 4,937,076 to Lapidus, discloses a chewable aspirin and buffering material tablet in a single dosage form. The buffering materials are integrally dispersed and bound in a fatty material of chocolate, synthetic chocolate or hydrogenated tallow. The fatty material individually coats the aspirin and buffering material.

U.S. Pat. No. 4,684,534 to Valentine discloses quick-liquefying, chewable tablets. The tablets have a harder outer shell which inhibits penetration of liquid, and a softer interior which quickly liquefies when the tablet and shell are broken into pieces and contacted by the liquid. The excipient or base material of the tablet is made from carbohydrates held together with small quantities of a carbohydrate binder such as maltodextrin. The tablets can contain active ingredients such as pharmaceuticals, breath sweeteners, vitamins and dietary supplements.

U.S. Pat. No. 4,609,543 to Morris et. Al. discloses a soft homogeneous antacid tablet. The tablet contains solid antacid particles thoroughly coated with a mixture composed of a fatty material or oil, a surfactant, and a flavor. The fat or oil is present in an amount of from about 25% to about 45% of the mixture. The primary particle size of the antacid is less than 100 millimicrons.

U.S. Pat. No. 4,446,135 to Valentine, discloses chewable calcium carbonate-containing antacid tablets having good mouth feel properties. The good mouth feel properties of the tablet are obtained by using calcium carbonate of a particular particle size in combination with certain excipients. The calcium carbonate is present in an effective amount and has a size from about 5 to 50 microns in diameter.

U.S. Pat. No. 5,320,848 to Geyer et. al. discloses a non-aqueous chewable composition for oral delivery of unpalatable drugs. The drug is intimately dispersed or dissolved in a pharmaceutically-acceptable lipid that is solid at room temperatures. The lipid material desirably readily melts with the application of mild temperature, i.e., from about 75° to about 95° C.

U.S. Pat. No. 5,837,285 to Nakamichi et. al. discloses fast soluble tablets that can be produced by a simple method. The tablet base is a sugar alcohol. The mixture of the sugar alcohol and a drug is subjected to compressive shaping prior to drying in the process. The dry solid tablet can be produced by modification of conventional tableting technology and possesses physico-chemical stability.

U.S. Pat. No. 4,446,135 to Fontaine, discloses chewable calcium carbonate-containing antacid tablets having good mouth feel properties. The good mouth feel properties of the tablet are obtained by using calcium carbonate of a particular particle size in combination with certain excipients. The calcium carbonate is present in an effective amount and has a size from about 5 to 50 microns in diameter.

U.S. Pat. No. 4,327,077 to Puglia et al. discloses a compressed chewable antacid tablet which has good flexibility, is breakage resistant and disintegrates immediately upon chewing. The tablet is formed of a re-crystallized fatty material, such as chocolate, a bulking material and an active ingredient bound up in the particles of the re-crystallized fatty material. The preferred re-crystallized fatty material is a chocolate or a synthetic chocolate.

U.S. Pat. No. 4,327,076, to Puglia et al., also discloses a compressed chewable antacid tablet which has good flexibility, is breakage resistant and disintegrates immediately upon chewing. The tablet is formed of particles of the antacid or other active ingredients which are admixed with particles formed of edible fat or oil absorbed on a fat-absorbing material, such as microcrystalline cellulose. Upon chewing, the tablet is quickly converted to a smooth creamy non-gritty palatable emulsion.

U.S. Pat. No. 5,320,848 to Geyer et. al. discloses a non-aqueous chewable composition for oral delivery of unpalatable drugs. The drug is intimately dispersed or dissolved in a pharmaceutically-acceptable lipid that is solid at room temperatures. The lipid material desirably readily melts with the application of mild temperatures, i.e. about 55 to 95 C.

However, the prior art compositions contain various disadvantages. For example, some tablet formulations may be incompletely chewed due to the poor palatability of the composition. Such compositions may also have a gummy texture, and are subject to “taste fatigue,” i.e., the composition is perceived to be less palatable after ingestion of multiple doses. Further, the binders and other materials used in such chewable tablets may prevent rapid and effective delivery of active materials to the stomach.

There is a need for a rapid-melt, composition that has a lubricious, palatable mouth-feel that behaves like a liquid when consumed and yet acts like a solid in many other ways. The need encompasses those compositions in which little to substantially no biting or chewing is necessary in order to cause the composition to melt, disintegrate, decompose, or otherwise break down or apart in the mouth. Such compositions are ideal for uses in the fields of pediatric and geriatric care, that is, for use with people or domestic mammals that do not have any teeth. These compositions are particularly useful for pediatric, geriatric patients or for those with limited ability to swallow traditional dosage forms.

Controlled—release pharmaceutical compositions are well known and described in the prior art. These generally comprise (a) at least one pharmaceutically active substance and one or more polymers, hydrating agents, dissolution agents, bulking agents, encapsulating agents and the like. Suitable controlled release polymers generally comprise a first polymer component and one or more secondary polymer components having different dissolution and drug wettability and release-ability characteristics from that of the first polymer. Known polymers useful in drug delivery systems may be soluble, insoluble, permeable, impermeable or biodegradable according to the physiological environment they are in. These may also be natural or synthetic polymers or copolymers.

### SUMMARY OF THE INVENTION

The present invention comprises a controlled-release oral tablet which allows for the long term administration of a pharmaceutical active. More specifically, the present invention is directed to the preparation of a controlled-release oral tablet comprising one or more long acting pharmaceutical salts and combinations thereof in a formulation to decrease the number of doses necessary to provide relief for bacterial and virus-related cold-flu-cough and sore throat oral symptoms. To this end, the present invention preferably comprises an orally dissolvable, controlled release two-layer tablet for the administration of an mineral active such as zinc, magnesium, copper and the like for sustained periods for enhanced efficacy in the treatment and relief of from cold flu and symptoms. More specifically, the present invention comprises a rapid melt, long acting bi-layered tablet containing high levels of an active compound encapsulated within bioadhesive/muco-adhesive polymers as a controlled release oral drug delivery system. The composition allows for increased residence time for enhanced prophylactic and therapeutic efficacy within the mouth and oral cavity. This allows for a reduction in the number of doses necessary to achieve therapeutic relief which will invariably result in increased patience compliance.

### DETAILED DESCRIPTION OF THE DRAWINGS

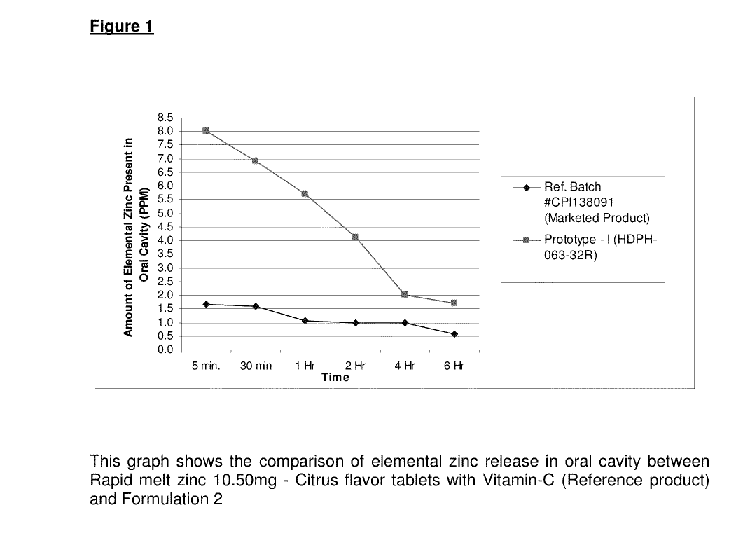

FIG. 1 is a graphic comparison of elemental zinc release in oral cavity between Rapid melt zinc 10.50 mg—Citrus flavor tablets with Vitamin-C (Reference product) and Formulation 2.

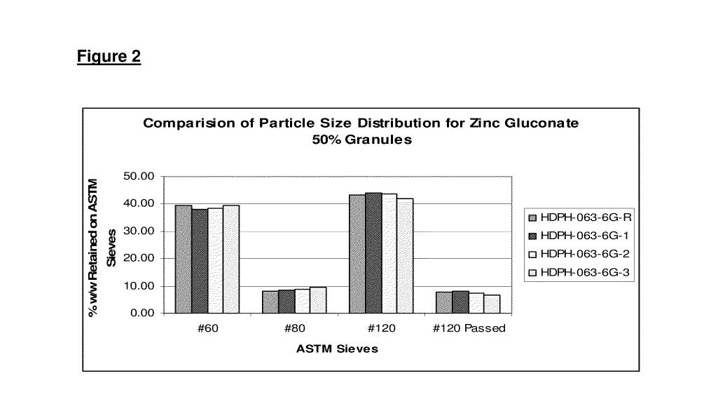

FIG. 2 is a graphic comparison of particle size distribution of various batches of 50% zinc gluconate granules.

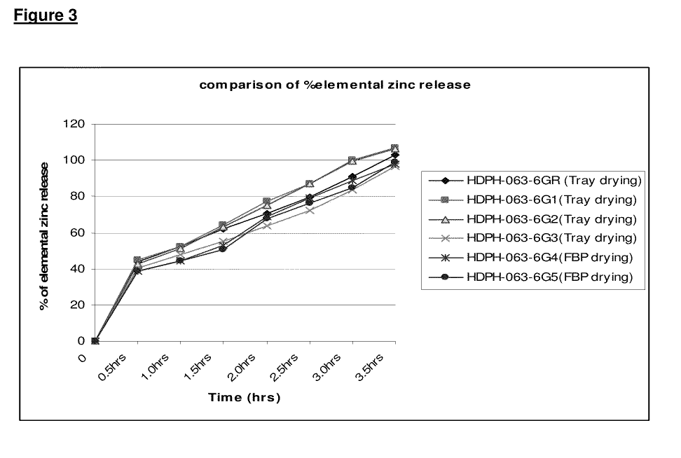

FIG. 3 is a graphic comparison of the percent (%) elemental zinc release in dissolution at different time points for various batches of 50% zinc gluconate granules.

### DETAILED DESCRIPTION OF THE INVENTION

Cough and cold/flu preparations for the delivery of an active ingredient for the treatment of symptoms related thereto have been in existence for many years and are well known in the art. These generally are dosage forms prepared as a hard candy, oral sprays, nasal sprays, orally disintegrating tablets (ODT) rapid melt dosage tablets, oral films, liquids and nasal swabs. These formulations work in the human body after administration by adhering to the mucosal lining of the mouth and oral cavity such as the cheeks, gums, upper throat, tongue and palate. In this way, the rapid melt formulations of the present invention provide continual therapeutic and prophylactic care. The rapid melt tablets can deliver any one of a number of mineral agents and their salts such as zinc, copper, manganese and the like in combination with flavoring agents, breath-freshening agents and the like.

The rapid melt, controlled release drug delivery system of the present invention may also contain and may be used for the administration of any one of a number of pharmaceutical actives selected from the group consisting of anti-bacterial agents, anti-tussives, expectorants, anti-viral agents, anti-inflammatory agents, neurotherapeutic agents, analgesics, anti-fungal agents, anti-neoplastic agents, anti-histamines, proteins, enzymes, minerals, hormonal agents, non-steroidal anti-inflammatories, cytokines, steroids, nicotine, insulin and mixtures thereof. Other such pharmaceutical actives include the following: antitussives, antihistamines, decongestants, alkaloids, mineral supplements, laxatives, vitamins, antacids, ion exchange resins, anti-cholesterolemics, anti-arrhythmics, anti-pyretics, analgesics, appetite suppressants, expectorants, anti-anxiety agents, anti-ulcer agents, anti-inflammatory substances, coronary dilators, cerebral dilators, peripheral vasodilators, anti-infectives, psycho-tropics, anti-maniocs, stimulants, gastrointestinal agents, sedatives, anti-diarrheal preparations, anti-angina drugs, vasodilators, anti-hypertensive drugs, vasoconstrictors, migraine treatments, antibiotics, tranquilizers, anti-psychotics, antitumor drugs, anticoagulants, antithrombotic drugs, hypnotics, anti-emetics, anti-nauseates, anti-consultants, neuromuscular drugs, hyper- and hypo-glycolic, spasmodic, uterine relaxants, mineral and nutritional additives, anti-obesity drugs, anabolic drugs, erythropoietin drugs, anti-asthmatics, cough suppressants, mucolytics, anti-uricemic drugs and mixtures thereof. Essentially any pharmaceutical active that may be solubilized and absorbed through the lining of the mouth is useful in the controlled delivery systems of the present invention.

The anhydrous nature of the present inventive compositions allows for very high doses of active materials to be incorporated therein. The amount of active material present in the inventive compositions will vary depending on the particular active used, but generally will be present in an amount of about 0.001% to 70% by weight of the composition
Preferably, the active is an organic or inorganic salt of a mineral such as zinc useful in the treatment of flu and cold symptoms. Suitable inorganic zinc salts include zinc bromide, zinc chloride, zinc iodine, zinc fluoride, zinc ammonium sulfate, zinc chromate, zinc fluorosilicate, zinc dithionate, zinc sulfate, zinc nitrate, zinc phosphate, zinc fluorozirconate, zinc oxide, and mixtures thereof. Suitable organic zinc salts include zinc citrate, zinc acetate, zinc gluconate, zinc asparate, zinc ascorbate, zinc ornate, zinc divalent amino acid zinc salts, zinc succinate, zinc tartrate, zinc glycerophosphate, zinc salicylate, zinc formate, and mixtures thereof. Preferably, the zinc salts utilized in the formulations of the present invention are the organic salts selected from the group comprising include zinc acetate, zinc gluconate, and mixtures thereof.

There is a definitive need then, for a cold/flu/cough preparation and formulation for the long term, controlled release of zinc, its salts, and combination with other actives, flavors, spices wherein the release of the active zinc compound will only occur during the lozenge or tablets residence time in the oral cavity. By providing a long term, controlled release of the active(s) in the oral cavity, more of the active can be absorbed through the epithelial mucosa. By utilizing this improved active delivery delivery system, one can reduce the number of doses needed to achieve the same therapeutic efficacy to reduce and eliminate the cold/flu/cough symptoms which will invariably increase patient compliance and result in better health and well being.

Without being bound to any theory, the present invention then comprises a novel, rapid-melt composition wherein the pharmaceutical active is first granulated with and micro-encapsulated by a carrier selected from the group consisting of bioadhesive/muco-adhesive polymers, biopolymers, fats, waxes, gums and mixtures thereof. The microencapsulated active is then further incorporated within a fast dissolving polymer matrix selected from the group consisting of cellulose and cellulose derivatives, thermoplastic polymers, hydrogels, gums and mixtures which are then granulated with the pharmaceutical active microencapsulated with the bioadhesive/muco-adhesive. Formulated as a fast-dissolving tablet with additional excipients such as sweeteners, flavors, binders, tabletting agents etc., the composition may be placed in the oral cavity for immediate dispersion/release of the components. Once released from the granulated fast melt, bioadhesive/muco-adhesive polymers adhere to all parts of the oral cavity including the oral epithelium, tongue, teeth, throat and esophagus. By retaining the pharmaceutical active for longer periods through mucosal adhesion, the active such as zinc acetate and zinc gluconate has a longer residence time to come into contact with bacteria and viruses and is more effective in the relief of the cold symptoms.

The controlled sustained release tablets of the present invention are prepared through the incorporation of microencapsulated zinc granules or spheres with one or more swelling hydratable muco-adhesive polymers, fats/waxes, gums and cellulose derivatives. These are then formulated into an rapid dissolving oral tablets comprised of one or more layers. Each layer can be formulated as a single release profile for delivery of the active or as a blend of fast and slow release granules wherein the active is released at different rates over time. The rapid-melt compositions are formed by molding or compression, with an additional heating step being preferred.

Because of the need for a smooth and relatively immobile surface for the placement and adherence of a muco-adhesive dosage form, the oral or buccal region appears to be more suitable for sustained delivery of therapeutic agents using muco-adhesive systems. The oral/buccal and sublingual routes for drug delivery are particularly advantageous since they avoid first pass metabolism. These regions consist of a non-keratinized epithelium, resulting in a somewhat more permeable tissue than the skin. Therefore, drugs with a short biological half-life requiring a sustained release effect and exhibiting poor permeability, sensitivity to enzymatic degradation, or poor solubility may be good candidates to be delivered via the oral cavity. Relevant muco-adhesive dosage forms for the oral cavity include gels, patches, tablets, and ointments.

Cold remedy fast melting tablets (Rapid melt zinc 10.50 mg bi-layered tablets with vitamin-C-orange flavor). are bi-layered round lozenge shaped tablets, comprised of white colored layer and another orange colored layer. Both layers (white and orange layer) contain zinc acetate and zinc gluconate as source of elemental zinc. The colored layer (orange layer) contains Vitamin-C along with zinc acetate and zinc gluconate. The pharmaceutical active such as an inorganic or organic salt of zinc may be incorporated in the tablets in amounts of from about 5.0 to about 25.0 mgs., and preferably in amounts of from about 10.0 to about 15.0 mgs.

Bioadhesive/muco-adhesive polymers are one of two (2) different types. The muco-adhesive polymers are water-soluble polymers selected from the group comprising synthetic polymers such as:

A. Cellulose derivatives selected from the group consisting of methylcellulose, ethyl cellulose, hydroxy-ethylcellulose, hydroxylpropyl cellulose, hydroxy propyl methylcellulose, sodium carboxy methylcellulose, poly(acrylic acid) polymers (carbomers, polycarbophil), poly(hydroxyethyl methylacrylate), poly(ethylene oxide), poly(vinyl pyrrolidone), poly(vinyl alcohol), natural polymers, tragacanth, sodium alginate, karaya gum, guar gum, xanthan gum, lecithin, soluble starch, gelatin, pectin, chitosan and mixtures.
B. Useful hydrophilic polymers include the water-soluble polymers that swell indefinitely in contact with water and eventually undergo complete dissolution, e.g. methyl cellulose, hydroxylethyl ellulose, hydroxylpropyl methyl cellulose, sodium carboxy methyl cellulose, carbomers, chitosan and plant gums. Hydrogels are water swellable materials, usually a cross-linked polymer with limited swelling capacity, e.g. poly (acrylic acid/acrylamide) copolymers, carrageenan, sodium alginate, guar gum and modified guar gum, etc.

Thermoplastic polymers include the non-erodible neutral polystyrene and semi-crystalline bio erodible polymers, which generate the carboxylic acid groups as they degrade, e.g. polyanhydrides and polylactic acid. Various synthetic polymers used in muco-adhesive formulations include polyvinyl alcohol, polyamides, polycarbonates, polyalkylene glycols, polyvinyl ethers, esters and halides, polymethacrylic acid, polymethylmethacrylic acid, Methyl Cellulose, hydroxylpropyl cellulose, hydroxylpropyl methyl cellulose, and sodium carboxy methyl cellulose.

Various bio-compatible polymers used in muco-adhesive formulations include cellulose-based polymers, ethylene glycol polymers and its copolymers, oxyethylene polymers, polyvinyl alcohol, polyvinyl acetate and esters of hyaluronic acid. Various biodegradable polymers useful in muco-adhesive formulations are poly (lactides), poly (glycolides), poly (lactide-co-glycolides), polycaprolactones, and polyalkyl cyanoacrylates. Polyorthoesters, polyphosphoesters, polyanhydrides, polyphosphazenes are the recent additions to the polymers.

In particular, the present invention comprises muco-adhesive polymers blended with zinc as the active ingredient. More particularly, the controlled release oral tablet is comprised of a sustained release layer (colored layer) comprising zinc gluconate, dry blended with muco-adhesive polymers selected from the group of cellulose derivatives (methylcellulose, ethyl cellulose, hydroxy-ethylcellulose, hydroxyl-propyl cellulose, hydroxy propyl methylcellulose, sodium carboxy methylcellulose, poly(acrylic acid) polymers (carbomers, polycarbophil), poly(hydroxyethyl methylacrylate), poly (ethylene oxide), Poly (vinyl pyrrolidone), poly (vinyl alcohol), natural polymers, Tragacanth, sodium alginate, karaya gum, guar gum, xanthan gum, lecithin, soluble starch, gelatin, pectin, chitosan. and mixtures thereof.

Natural polymers include polypeptides, polysaccharides and alginic acid. Well known pharmaceutically acceptable polymers include peptides and polysaccharides. Representative synthetic polymers include alkyl celluloses, hydroxyalkyl celluloses, cellulose ethers, cellulose esters, nitro celluloses, polymers of acrylic and methacrylic acids and esters thereof, polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes and polyurethanes and co-polymers thereof. The polymer to be used is governed by its toxicity and its compatability with the particular active ingredient being used and can be selected without difficulty by those skilled in the art.

Particularly suitable polymers include: methyl cellulose, ethyl cellulose, hydroxypropyl cellulose, hydroxypropyl methyl cellulose, hydroxybutyl methyl cellulose, cellulose acetate, cellulose propionate (lower, medium or higher molecular weight), cellulose acetate propionate, cellulose acetate butyrate, cellulose acetate phthalate, carboxymethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene glycol), poly(ethylene oxide), poly(ethylene terephthalate), poly(vinyl alcohol), poly(vinyl isobutyl ether), poly(vinyl acetate), poly(vinyl chloride) and polyvinylpyrrolidone.

Especially suitable co-polymers include: butyl methacrylate/isobutyl methacrylate co-polymer, high molecular weight, methylvinyl ether/maleic acid co-polymer, methylvinyl ether/maleic acid, monoethyl ester co-polymer, methylvinyl ether/maleic anhydride co-polymer and vinyl alcohol/vinyl acetate co-polymer. Representative biodegradable polymers include, polylactides, polyglycolides, poly(ethylene terephthalate) and polyurethane. The rapid-melt composition of the present inventive subject matter also contains a salivating agent. As is used herein, “salivating agent” means a material that promotes greater salivation in the user of the compositions of the present inventive subject matter. The salivating agent helps create salivation in the mouth of the mammal using the inventive compositions. This is an important feature since the present compositions are intended to be taken by the patient without the aid of water to help in the transporting of the composition to the stomach of the patient. The salivating agent can be, without limitation, an emulsifier or a food acid that initiates salivation in the mouth of the patient.

Examples of emulsifiers useful as salivating agents in the compositions of the present inventive subject matter include, without limitation, alkyl aryl sulfonates, alkyl sulfates, sulfonated amides and amines, sulfated and sulfonated esters and ethers, alkyl sulfonates, polyethoxylated esters, mono-, di-, and triglycerides, diacetyl tartaric esters of monoglycerides, polyglycerol esters, sorbitan esters and ethoxylates, lactylated esters, phospholipids such as lecithin, polyoxyethylene sorbitan esters, propylene glycol esters, sucrose esters, and mixtures thereof. The emulsifier may be either saturated or unsaturated. It should be noted that some of the emulsifiers that are salivating agents may also function as binders.

Examples of food acids useful as salivating agents in the inventive compositions include, without limitation, citric acid, malic acid, tartarate, food salts such as sodium chloride and salt substitutes, potassium chloride, and mixtures thereof.

The amount of salivating agent present in the rapid-melt composition of the present inventive subject matter is from about 0.05% to about 15% by weight of the final composition. Preferably, the amount of salivating agent from about 0.3% to 0.4% by weight of the composition.

Keeping the amount of salivating agent present in the inventive composition within these limits for weight percentage is important to enhance the desirable properties of the compositions. More particularly, the low amount of salivating agent present in the compositions aid in the compositions retaining the physical state and the rapidity of melting in the mouth of a mammal.

The rapid-melt compositions of the present inventive subject matter further contain a diluent/bulking material. The use of a diluent/bulking material is necessary to serve as a free-flow imparting agent which aids in the moisturizing of the composition when chewed, that is, the diluent/bulking material aids in the processability of the compositions. The diluent/bulking material also serves to reduce the concentration of the active materials and add bulk to the composition.

Examples of diluent/bulking materials useful in the compositions of the present inventive subject matter include, without limitation, silicon dioxide, sugars, starches, lactose, sucrose, sorbitol, fructose, talc, stearic acid, magnesium stearate, dicalcium phosphate, erythitol, xylitol, mannitol, maltitol, isomalt, dextrose, maltose, lactose, microcrystalline celluloses and mixtures thereof. It should be noted that some of the diluents/bulking materials also function as binders. The amount of diluent/bulking material present in the compositions is from about 0.5% to about 99% by weight of the final composition. Preferably, the amount of diluent/bulking material is from about 2% to about 95% by weight of the composition.

The rapid-melt compositions of the present inventive subject matter may optionally contain a further slipping agent to aid in the palatability of the composition after it melts in the mouth of the mammal. The slipping agent may be a further lipid material, as is described above for binders, or another material which aids in the “slipping” of the composition through the mouth and down the esophagus of the mammal.

The rapid-melt compositions of the present inventive subject matter contain at least one binder. As used herein, “binder” means at least one ingredient useful in keeping the composition in its state, may be either solid or liquid, and may include, without limitation, a high melting point fat or waxy material such as lipid materials, polyethylene glycols (PEG), waxes and other fats. Preferably, the compositions of the present inventive subject matter contains a mixture of binders. The solid binders useful in the compositions of the present inventive subject matter have a melting point of about 25 to 90° C., and preferably about 37° C. When more than one binder is used in the inventive compositions, the melting point of the combination of the binders will remain within the range of 25 to 90° C., and preferably about 37° C. The inventive subject matter contemplates the use of mixtures of solid binders and liquid binders. For a non-limiting example, the present inventive subject matter contemplates mixing a small amount of a high-melting point lipid with a liquid binder to achieve a binder that attains the desired product characteristics. These characteristics include such factors as mouth feel, rapidity of melting in the mouth, appearance, flavor and compatibility with active materials and therapeutic active materials.

The rapid-melt compositions of the present inventive subject matter contain at least one binder. As used herein, “binder” means at least one ingredient useful in keeping the composition in its state, may be either solid or liquid, and may include, without limitation, a high melting point fat or waxy material such as lipid materials, polyethylene glycols (PEG), waxes and other fats. Preferably, the compositions of the present inventive subject matter contains a mixture of binders. The solid binders useful in the compositions of the present inventive subject matter have a melting point of about 25 to 90.degree. C., and preferably about 37.degree. C. When more than one binder is used in the inventive compositions, the melting point of the combination of the binders will remain within the range of 25 to 90° C., and preferably about 37.degree. C. The inventive subject matter contemplates the use of mixtures of solid binders and liquid binders. For a non-limiting example, the present inventive subject matter contemplates mixing a small amount of a high-melting point lipid with a liquid binder to achieve a binder that attains the desired product characteristics. These characteristics include such factors as mouth feel, rapidity of melting in the mouth, appearance, flavor and compatibility with active materials and therapeutic active materials.

Among the lipid materials useful as binders in the compositions of the present inventive subject matter are those which are commercially available and commonly used in confectionery and other food products. Such lipid materials include, without limitation, cocoa butter, hydrogenated tallow, hydrogenated vegetable oils, hydrogenated cotton seed oil, palm kernel oil, soybean oil, stannol esters, and derivatives and mixtures thereof. Hydrogenated vegetable oils (such as hydrogenated palm kernel oil), cocoa butter, and cocoa butter substitutes are among the preferred useful lipid materials. Additional binders may include emulsifiers, surface active agents, plasticizers, such as glycerol esters, polyalcohol esters, polyoxyethylene esters of hydrophilic and hydrophobic balances from 0.5 to above 20 and polyethylene glycols. Other examples include saccharides such as monosaccharides and oligosaccharides. Examples of monosaccahrides include: dextrose, dextrose monohydrate, lactose, mannose, fructose, etc. Liquid binders may also be used. Examples of liquid binders are, without limitation, polysaccharides, gum solutions, water, corn syrup, hydrogenated starch hydrolysates, glycerine, polypropylene glycol, polyethylene glycols, and mixtures thereof. It should be noted that liquid binders, when used may be present in quantities to not affect the constituency of the product so that the final product retains a predominantly solid constituency. In some aspects, the liquid binders may not exceed about 5% of the composition.

Among the lipid materials particularly useful as binders in the compositions of the present inventive subject matter are those which are commercially available and commonly used in confectionery and other food products. Such lipid materials include, without limitation, cocoa butter, hydrogenated tallow, hydrogenated vegetable oils, hydrogenated cotton seed oil, palm kernel oil, soybean oil, stannol esters, and derivatives and mixtures thereof. Hydrogenated vegetable oils (such as hydrogenated palm kernel oil), cocoa butter, and cocoa butter substitutes are among the preferred useful lipid materials. Additional binders may include glycerol esters, polyalcohol esters, polyoxyethylelne esters of hydrophilic and hydrophobic balances from 0.5 to above 20 and polyethylene glycols. Other examples include saccharides such as monosaccharides and oligosaccharides. Examples of monosaccahrides include: dextrose, dextrose monohydrate, lactose, mannose, fructose, etc. Liquid binders may also be used. Examples of liquid binders are, without limitation, polysaccharides, gum solutions, water, corn syrup, hydrogenated starch hydrolates, glycerine, polypropylene glycol, and mixtures thereof. It should be noted that liquid binders, when used may be present in quantities to not affect the constituency of the product so that the final product retains a predominantly solid constituency. In some aspects, the liquid binders may not exceed about 5% of the composition.

The amount of binder present in the rapid-melt composition of the present inventive subject matter is from about 0.01% to about 70% by weight of the final composition. Preferably, the amount of binder is from about 0.01% to about 50% by weight of the composition. More preferably the binder is present from about 15% to about 30% by weight of the composition.

The binder is used to provide good melt away properties to the composition while preventing a gritty texture being imparted by the composition. The binder aids in the fast melting of the composition when placed in the mouth of a user.

The rapid-melt composition of the present inventive subject matter also contains a salivating agent. As is used herein, “salivating agent” means a material that promotes greater salivation in the user of the compositions of the present inventive subject matter. The salivating agent helps create salivation in the mouth of the mammal using the inventive compositions. This is an important feature since the present compositions are intended to be taken by the patient without the aid of water to help in the transporting of the composition to the stomach of the patient. The salivating agent can be, without limitation, an emulsifier or a food acid that initiates salivation in the mouth of the patient.

Examples of emulsifiers useful as salivating agents in the compositions of the present inventive subject matter include, without limitation, alkyl aryl sulfonates, alkyl sulfates, sulfonated amides and amines, sulfated and sulfonated esters and ethers, alkyl sulfonates, polyethoxylated esters, mono-, di-, and triglycerides, diacetyl tartaric esters of monoglycerides, polyglycerol esters, sorbitan esters and ethoxylates, lactylated esters, phospholipids such as lecithin, polyoxyethylene sorbitan esters, proplyene glycol esters, sucrose esters, and mixtures thereof. The emulsifier may be either saturated or unsaturated. It should be noted that some of the emulsifiers that are salivating agents may also function as binders.

The rapid-melt compositions of the present inventive subject matter contain at least one binder. As used herein, “binder” means at least one ingredient useful in keeping the composition in its state, may be either solid or liquid, and may include, without limitation, a high melting point fat or waxy material such as lipid materials, polyethylene glycols (PEG), waxes and other fats. Preferably, the compositions of the present inventive subject matter contains a mixture of binders. The solid binders useful in the compositions of the present inventive subject matter have a melting point of about 25 to 90.degree. C., and preferably about 37.degree. C. When more than one binder is used in the inventive compositions, the melting point of the combination of the binders will remain within the range of 25 to 90.degree. C., and preferably about 37.degree. C. The inventive subject matter contemplates the use of mixtures of solid binders and liquid binders. For a non-limiting example, the present inventive subject matter contemplates mixing a small amount of a high-melting point lipid with a liquid binder to achieve a binder that attains the desired product characteristics. These characteristics include such factors as mouth feel, rapidity of melting in the mouth, appearance, flavor and compatibility with active materials and therapeutic active materials.
Among the lipid materials useful as binders in the compositions of the present inventive subject matter are those which are commercially available and commonly used in confectionery and other food products. Such lipid materials include, without limitation, cocoa butter, hydrogenated tallow, hydrogenated vegetable oils, hydrogenated cotton seed oil, palm kernel oil, soybean oil, stannol esters, and derivatives and mixtures thereof. Hydrogenated vegetable oils (such as hydrogenated palm kernel oil), cocoa butter, and cocoa butter substitutes are among the preferred useful lipid materials. Additional binders may include emulsifiers, surface active agents, plasticizers, such as glycerol esters, polyalcohol esters, polyoxyethylene esters of hydrophilic and hydrophobic balances from 0.5 to above 20 and polyethylene glycols. Other examples include saccharides such as monosaccharides and oligosaccharides. Examples of monosaccahrides include: dextrose, dextrose monohydrate, lactose, mannose, fructose, etc. Liquid binders may also be used. Examples of liquid binders are, without limitation, polysaccharides, gum solutions, water, corn syrup, hydrogenated starch hydrolysates, glycerine, polypropylene glycol, polyethylene glycols, and mixtures thereof. It should be noted that liquid binders, when used may be present in quantities to not affect the constituency of the product so that the final product retains a predominantly solid constituency. In some aspects, the liquid binders may not exceed about 5% of the composition.
The following examples are provided to more specifically set forth and define the process of the present invention. It is recognized that changes may be made to the specific parameters and ranges disclosed herein and that there are a number of different ways known in the art to change the disclosed variables. And whereas it is understood that only the preferred embodiments of these elements are disclosed herein as set forth in the specification and drawings, the invention should not be so limited and should be construed in terms of the spirit and scope of the claims that follow herein.

### Example 1

The quantitative bio-analysis data of a rapid melt zinc 14.5 mg long acting bi-layered tablets with orange flavor was compared with a rapid melt zinc 10.50 mg tablets with Vitamin-C-Orange flavor. A two-layer tablet was comprised of a first layer (white layer) that contained both a zinc acetate and a zinc gluconate active equals to 5.25 mg elemental zinc. A second layer of the tablet (the orange layer) contains both actives in an amount that equals to 5.25 mg elemental Zinc along with Vitamin-C. The formulations comprising the actives are set forth in Table 1 below.

TABLE 1
The following table shows the formulation variables.

| | Active variables | |
|---|---|---|
| | Zinc acetate + Zinc | |
| Product form | gluconate | Zinc gluconate |
| The Reference product: Rapid melt | 1^{st} layer (white layer) | None |
| zinc 10.50 mg tablets -Orange flavor | contains both actives | |
| with Vitamin-C | equivalent to 5.25 mg | |
| 1^{st} Layer (White layer): 410 mg white | elemental Zinc. | |
| layer contains both actives. | 2^{nd} layer (orange layer) | |
| 2^{nd} layer (Orange layer): 410 mg | contains both actives | |
| orange layer contains both actives | equals to 5.25 mg | |
| with vitamin C. | elemental Zinc along with | |
| | vitamin-C. | |
| Formulation A - Rapid melt zinc 14.50 mg. | 1^{st} layer (white layer) | 2^{nd} layer (orange |
| long acting bi-layered tablets- | contains both actives in | layer): Long |
| orange flavor. | 83:17 ratio equivalent to | acting layer |
| 1^{st} Layer (White layer): 450 mg. white | 9.50 mg Zinc. | contains 50% zinc |
| layer contains both actives. | | gluconate CR |
| 2^{nd} layer (Orange layer): 450 mg | | granules equals |
| orange layer contains 50% zinc | | to 5 mg Zinc. |
| gluconate controlled release (CR) | | |
| granules. | | |
| Formulation B - Rapid melt zinc 14.50 mg | 1^{st} layer (white layer) | 2^{nd} layer (orange |
| long acting bi-layered tablets- | contains zinc acetate di- | layer): Long |
| orange flavor. | hydrate equals to 4.50 mg | acting layer |
| 1^{st} Layer (White layer): 450 mg white | zinc and zinc | contains 50% zinc |
| layer contains both zinc actives. | gluconate 50% CR | gluconate CR |
| 2^{nd} layer (Orange layer): 450 mg | granules equals to | granules equals |
| orange layer contains 50% zinc | 5.00 mg Zinc. | to 5 mg Zinc. |
| gluconate CR granules. | | |

Tabletting Process:
Bi-layered tablets are compressed using a ten (10) stations pilot press by individually weighing the two layers (Rapid melt layer + long acting layer) and manually poured into the die then compressed into the bi-layered tablets using one upper punch and one lower punch.

### Example 2

| | | Elemental Zinc release bio analytical data with | | | | | |
|---|---|---|---|---|---|---|---|
| Ex. | Product | respect to Time points (PPM) | | | | | |
| No. | type | 5 mins | 30 mins | 1 hr | 2 hr | 4 hr | 6 hr |
| 1 | Reference | 1.65 | 1.6 | 1.05 | 1 | 1 | 0.55 |
| | (Marketed | | | | | | |
| | Product) | | | | | | |
| 2 | Formu- | 8 | 6.9 | 5.7 | 4.1 | 2 | 1.7 |
| | lation A: | | | | | | |
| 3 | % | 79.38% | 76.81% | 81.58% | 75.61% | 50% | 67.65% |
| | Difference | more | more | more | more | more | more |
| | of | | | | | | |
| | elemental | | | | | | |
| | zinc | | | | | | |
| | release in | | | | | | |
| | oral cavity | | | | | | |
| | compare | | | | | | |
| | to | | | | | | |
| | Reference | | | | | | |
| | product | | | | | | |

A quantitative analysis was carried out to compare the amount of elemental zinc release in oral cavity from Rapid melt zinc 10.50 mg—Citrus flavor tablets with Vitamin-C (Reference tablet) (Marketed product) with Formulation-I (B#: HDPH-063-31R).
The results clearly show that much more of the elemental zinc administered by the two-layered tablet of the present invention (prototype) is present in the oral cavity over a six (6) hour period than the amount of zinc measured after administration of a formulation known in the art.

### Example 3

A qualitative bio-analysis was conducted wherein the elemental zinc that was present in the oral cavity was measured by collecting the saliva at different time points from clinical participants who had orally ingested inventive formulations 1 and 2. Saliva samples were taken from a number of volunteer subjects by swabbing the oral cavity of each individual after ingestion of the reference standard formulation and those of the claimed invention. in each case, a 0.01% dithizone reagent was used to indicate the presence of the zinc compounds in the oral cavity. The 0.01% dithizone reagent indicator was prepared by accurately weighing about 5.0 mg of dithizone in 50 ml of carbon tetrachloride which was the shaken well until it is completely dissolved. The subjects were not allowed any kind of food, alcohol, drink, smoking and any medications prior to 3 hrs of sampling which was continued until the sampling is completed. Each subject rinsed their mouths before taking the sample with water and the pH of saliva in each subject prior to start the experiment

| Product form | | Batch No. |
|---|---|---|
| 2.1 | Rapid melt Zinc 10.5 mg bi-layered Tablets- | Reference |
| | Orange Flavor. | |
| 2.2 | Rapid melts Zinc 14.5 mg long acting bi- | Formulation 1 |
| | layered Tablets-Orange Flavor. | (Zinc Gluconate CR |
| | | granules Batch no: |
| | | HDPH-063-6G-5) |
| 2.3 | Rapid melts Zinc 14.5 mg long acting bi- | Formulation 2 |
| | layered Tablets-Orange Flavor. | |

### 3.0 Bio-Analytical Results: (Qualitative Analysis):

3.1 Color indication observations: Dithizone reagent it self having green color.
3.2 If the color changes from green to violet, it indicates the presence of Zinc in oral cavity.
3.3 If the green color does not change, it indicates absence of Zinc in oral cavity.
3.4 The following table shows the comparison of presence elemental zinc in the oral cavity at different time stations in 2 different subjects.

| | | pH | | | | | |
|---|---|---|---|---|---|---|---|
| | | of | Age/ | Presence of elemental zinc in oral cavity | | | |
| Product | Subject | saliva | gender | Blank | 1st hour | 3rd hour | 6th hour |
| 3.4.1. Rapid | Subject-1 | 7.2 | 25/Male | Green | Green | Green | Green color |
| melt Zinc | | | | color didn't | color | color | changed to |
| 10.5 mg bi- | | | | changed to | changed to | changed to | violet |
| layered Tablets- | | | | violet | violet | violet | (Zinc |
| Orange Flavor. | | | | (Zinc | (Zinc | (Zinc | present) |
| Reference | | | | absent0 | present) | present) | |
| Control | Subject-2 | 7.0 | 26/Male | Green | Green | Green | Green color |
| | | | | color didn't | color | color | changed to |
| | | | | changed to | changed to | changed to | violet |
| | | | | violet | violet | violet | (Zinc |
| | | | | (Zinc | (Zinc | (Zinc | presence) |
| | | | | absence) | presence) | presence) | |
| Formulation 1 | Subject-1 | 7.1 | 25/Male | Green | Green | Green | Green color |
| (Zinc Gluconate | | | | color didn't | color | color | changed to |
| CR granules | | | | changed to | changed to | changed to | violet |
| | | | | violet | violet | violet | (Zinc |
| | | | | (Zinc | (Zinc | (Zinc | presence) |
| | | | | absence) | presence) | presence) | |
| | Subject-2 | 7.0 | 26/Male | Green | Green | Green | Green color |
| | | | | color didn't | color | color | changed to |
| | | | | changed to | changed to | changed to | violet |
| | | | | violet | violet | violet | (Zinc |
| | | | | (Zinc | (Zinc | (Zinc | presence) |
| | | | | absence) | presence) | presence) | |
| Rapid Melt Zinc | Subject-1 | 7.0 | 25/Male | Green | Green | Green | Green color |
| 14.5 mg Long | | | | color didn't | color | color | changed to |
| Acting Bi- | | | | changed to | changed to | changed to | violet |
| Layered | | | | violet | violet | violet | (Zinc |
| Tablets-Orange | | | | (Zinc | (Zinc | (Zinc | presence) |
| Flavor | | | | absence) | presence) | presence) | |
| (B# HDPH-063- | Subject-2 | 7.0 | 26/Male | Green | Green | Green | Green color |
| 39) | | | | color didn't | color | color | changed to |
| (Zinc Gluconate | | | | changed to | changed to | changed to | violet |
| CR granules | | | | violet | violet | violet | (Zinc |
| Batch no: | | | | (Zinc | (Zinc | (Zinc | presence) |
| HDPH-063-6G- | | | | absence) | presence) | presence) | |
| 5) | | | | | | | |

### Conclusion:

The above qualitative bio-analytical results of both Rapid melt Zinc 10.5 mg bi-layered Tablets-Orange Flavor (Reference) and Rapid melt zinc 14.5 mg long acting bi-layer tablets-orange flavor (Formulations 1 and 2) shows that elemental zinc is present in oral cavity up to 6 hours. Hence the quantitative estimation of amount of elemental zinc present in the clinical subjects oral cavity is better for differentiation of Rapid melt zinc 10.5 mg bilayered tablets-orange flavor (Lot#CPI138091) verses Rapid melt zinc 14.50 mg long acting bilayered tablets-orange flavor (Inventive formulations 1 and 2).

### Example 4

Zinc gluconate granules used to formulate the Rapid melt Zinc 14.5 mg long acting bi-ilayered tablets with orange flavor were prepared and dried using tray drier (Batch A) and two batches (B & C) were dried using a fluid bed processor (FBP). Dissolution analysis was carried out using six batches of 50% zinc gluconate granules using following conditions. Based on solubility of zinc, water is selected as a dissolution medium.
Particle size distribution of 50% Zinc gluconate granules was identical for all the batches.
The following table shows a comparison of the amount of elemental zinc release based as a weight percent from 50% zinc granules at different time point's for six different batches.

| | % elemental zinc release | | | | | |
|---|---|---|---|---|---|---|
| | Granules are dried using Tray drier | | | | Granules are dried using | |
| | HDPH- | HDPH- | HDPH- | HDPH- | Fluid bed processor | |
| Time | 063- | 063- | 063- | 063- | HDPH-063- | HDPH- |
| (Hrs) | 6GR | 6G1 | 6G2 | 6G3 | 6G4 | 063-6G5 |
| 0.5 hrs | 43.80 | 44.87 | 42.74 | 40.60 | 38.43 | 38.43 |
| 1.0 hrs | 52.20 | 52.22 | 51.11 | 47.79 | 44.45 | 44.45 |
| 1.5 hrs | 62.19 | 64.44 | 63.28 | 55.37 | 52.98 | 50.78 |
| 2.0 hrs | 70.62 | 77.56 | 75.18 | 63.42 | 68.80 | 67.61 |
| 2.5 hrs | 79.70 | 87.10 | 86.91 | 72.05 | 78.87 | 76.45 |
| 3.0 hrs | 90.80 | 100.01 | 99.78 | 83.75 | 88.63 | 84.84 |
| 3.5 hrs | 103.0 | 106.72 | 106.48 | 96.75 | 98.08 | 98.91 |

It is evident from the above results that all six batches show prolonged zinc release and hence active action up to 3.0-3.5 hours. Moreover, all six batches also show an elemental zinc release of 45%-55% up to one (1.0) hour.
Six (6) batches show prolonged elemental zinc release of 60%-80% in up to 2.0 hours. and six batches are show an elemental zinc release of more than 80% in 3.0 hours.
The following examples teach suitable formulations that are useful in the practice of the present invention. Percentages given are based on a weight percent.

### Example 5

### a) Formula for Zinc Gluconate 50% Granules (Polymer: Drug Ratio is 1:4)

| Material Name | mg/dose |
|---|---|
| Active | |
| Zinc gluconate dihydrate USP | 75.19 |
| (75.19 * 13.30/100 = 10.00 mg) | |
| Bioadhesive | |
| Hydroxy propyl methyl cellulose | 18.80 |
| K100 M CR USP/EP | |
| Diluents | |
| Microcrystalline cellulose NF/Ph. Eur. | 56.39 |
| Solvent | |
| Purified water USP | q.s. |
| Total | 150.38 |

### b) Formula for Zinc Rapid Melt 14.5 mg Long Acting Tablets—Black Cherry Flavor

| Material Name | mg/tablet |
|---|---|
| Active | |
| Milled Zinc Acetate Dihydrate | 15.12 |
| (15.12 * 29.78/100 = 4.50 mg) | |
| Zinc gluconate dihydrate 50% granules | 150.38 |
| Diluents | |
| Mannitol Powder USP/NF | 45.00 |
| Mannitol Granules USP/NF | 360.09 |
| Microcrystalline Cellulose NF/Ph. Eur. | 198.00 |
| Disintegrants | |
| Crospovidone USP | 63.00 |
| Sod. Starch Glycolate NF | 18.00 |
| Emulsifiers | |
| Polysorbate 80 NF | 2.25 |
| Sodium Lauryl Sulfate NF | 0.90 |
| Sorbitan Monostearate | 4.50 |
| Binder | |
| Povidone USP/NF | 1.80 |
| Hardness Enhancer | |
| Polyethylene Glycol 8000 NF | 9.00 |
| Lubricants | |
| Magnesium Stearate NF | 7.20 |
| Purified talc USP | 3.60 |
| Silicon dioxide NF | 1.80 |
| Flavor | |
| Black Cherry Natural Sweet Type- | 11.70 |
| Flavor 207J98 | |
| Sweetener | |
| Sucralose NF | 4.50 |
| Acesulfame K FCC | 2.25 |
| Color | |
| FD&C Red No. 40 Aluminum Lake HT | 0.90 |
| Total | 900.00 |

### Example 6

### a) Formula for Zinc Gluconate 50% Granules (Polymer: Drug Ratio is 1:4)

| Material Name | mg/dose |
|---|---|
| Active | |
| Zinc gluconate dihydrate USP | 75.19 |
| (75.19 * 13.30/100 = 10.00 mg) | |
| Bioadhesive | |
| Hydroxy propyl methyl cellulose | 18.80 |
| K4 M CR USP/EP | |
| Diluents | |
| Microcrystalline cellulose NF/Ph. Eur. | 56.39 |
| Solvent | |
| Purified water USP | q.s. |
| Total | 150.38 |

### b) Formula for Zinc Rapid Melt 14.5 mg Long Acting Tablets—Black Cherry Flavor

| Material Name | mg/tablet |
|---|---|
| Active | |
| Milled Zinc Acetate Dihydrate | 15.12 |
| (15.12 * 29.78/100 = 4.50 mg) | |
| Zinc gluconate dihydrate 50% granules | 150.38 |
| Diluents | |
| Mannitol Powder USP/NF | 45.00 |
| Mannitol Granules USP/NF | 360.09 |
| Microcrystalline Cellulose NF/Ph. Eur. | 198.00 |
| Disintegrants | |
| Crospovidone USP | 63.00 |
| Sod. Starch Glycolate NF | 18.00 |
| Emulsifiers | |
| Polysorbate 80 NF | 2.25 |
| Sodium Lauryl Sulfate NF | 0.90 |
| Sorbitan Monostearate | 4.50 |
| Binder | |
| Povidone USP/NF | 1.80 |
| Hardness Enhancer | |
| Polyethylene Glycol 8000NF | 9.00 |
| Lubricants | |
| Magnesium Stearate NF | 7.20 |
| Purified talc USP | 3.60 |
| Silicon dioxide NF | 1.80 |
| Flavor | |
| Black Cherry Natural Sweet Type- | 11.70 |
| Flavor 207J98 | |
| Sweetener | |
| Sucralose NF | 4.50 |
| Acesulfame K FCC | 2.25 |
| Color | |
| FD&C Red No. 40 Aluminum Lake HT | 0.90 |
| Total | 900.00 |

### Example 7

### a) Formula for Zinc Gluconate 50% Granules (Polymer: Drug Ratio is 1:4)

| Material Name | mg/dose |
|---|---|
| Active | |
| Zinc gluconate dihydrate USP | 75.19 |
| (75.19 * 13.30/100 = 10.00 mg) | |
| Bioadhesive | |
| Hydroxy propyl methyl cellulose | 18.80 |
| K15 M CR USP/EP | |
| Diluents | |
| Microcrystalline cellulose NF/Ph. Eur. | 56.39 |
| Solvent | |
| Purified water USP | q.s. |
| Total | 150.38 |

### b) Formula for Zinc Rapid Melt 14.5 mg Long Acting Tablets—Black Cherr Flavor

| Material Name | mg/tablet |
|---|---|
| Active | |
| Milled Zinc Acetate Dihydrate | 15.12 |
| (15.12 * 29.78/100 = 4.50 mg) | |
| Zinc gluconate dihydrate 50% granules | 150.38 |
| Diluents | |
| Mannitol Powder USP/NF | 45.00 |
| Mannitol Granules USP/NF | 360.09 |
| Microcrystalline Cellulose NF/Ph. Eur. | 198.00 |
| Disintegrants | |
| Crospovidone USP | 63.00 |
| Sod. Starch Glycolate NF | 18.00 |
| Emulsifiers | |
| Polysorbate 80 NF | 2.25 |
| Sodium Lauryl Sulfate NF | 0.90 |
| Sorbitan Monostearate | 4.50 |
| Binder | |
| Povidone USP/NF | 1.80 |
| Hardness Enhancer | |
| Polyethylene Glycol 8000NF | 9.00 |
| Lubricants | |
| Magnesium•stearate NF | 7.20 |
| Purified talc USP | 3.60 |
| Silicon dioxide NF | 1.80 |
| Flavor | |
| Black Cherry Natural Sweet Type- | 11.70 |
| Flavor 207J98 | |
| Sweetener | |
| Sucralose NF | 4.50 |
| Acesulfame K FCC | 2.25 |
| Color | |
| FD&C Red No. 40 Aluminum Lake HT | 0.90 |
| Total | 900.00 |

### Example 8

### a) Formula for Zinc Gluconate 50% Granules (Polymer: Drug Ratio is 1:4)

| Material Name | mg/dose |
|---|---|
| Active | |
| Zinc gluconate dihydrate USP | 75.19 |
| (75.19 * 13.30/100 = 10.00 mg) | |
| Bioadhesive | |
| Hydroxy propyl methyl cellulose | 18.80 |
| K100 LV Premium USP/EP | |
| Diluents | |
| Microcrystalline cellulose NF/Ph. Eur. | 56.39 |
| Solvent | |
| Purified water USP | q.s. |
| Total | 150.38 |

### b) Formula for Zinc Rapid Melt 14.5 mg Long Acting Tablets—Black Cherr Flavor

| Material Name | mg/tablet |
|---|---|
| Active | |
| Milled Zinc Acetate Dihydrate | 15.12 |
| (15.12 * 29.78/100 = 4.50 mg) | |
| Zinc gluconate dihydrate 50% granules | 150.38 |
| Diluents | |
| Mannitol Powder USP/NF | 45.00 |
| Mannitol Granules USP/NF | 360.09 |
| Microcrystalline Cellulose NF/Ph. Eur. | 198.00 |
| Disintegrants | |
| Crospovidone USP | 63.00 |
| Sod. Starch Glycolate NF | 18.00 |
| Emulsifiers | |
| Polysorbate 80 NF | 2.25 |
| Sodium Lauryl Sulfate NF | 0.90 |
| Sorbitan Monostearate | 4.50 |
| Binder | |
| Povidone USP/NF | 1.80 |
| Hardness Enhancer | |
| Polyethylene Glycol 8000NF | 9.00 |
| Lubricants | |
| Magnesium Stearate NF | 7.20 |
| Purified talc USP | 3.60 |
| Silicon dioxide NF | 1.80 |
| Flavor | |
| Black Cherry Natural Sweet Type- | 11.70 |
| Flavor 207J98 | |
| Sweetener | |
| Sucralose NF | 4.50 |
| Acesulfame K FCC | 2.25 |
| Color | |
| FD&C Red No. 40 Aluminum Lake HT | 0.90 |
| Total | 900.00 |

### Example 9

### a) Formula for Zinc Gluconate 50% Granules (Polymer: Drug Ratio is 1:4)

| Material Name | mg/dose |
|---|---|
| Active | |
| Zinc gluconate dihydrate USP | 75.19 |
| (75.19 * 13.30/100 = 10.00 mg) | |
| Bioadhesive | |
| Polycarbophil USP | 18.80 |
| Diluents | |
| Microcrystalline cellulose NF/Ph. Eur. | 56.39 |
| Solvent | |
| Purified water USP | q.s. |
| Total | 150.38 |

### b) Formula for Zinc Rapid Melt 14.5 mg Long Acting Tablets—Black Cherr Flavor

| Material Name | mg/tablet |
|---|---|
| Active | |
| Milled Zinc Acetate Dihydrate | 15.12 |
| (15.12 * 29.78/100 = 4.50 mg) | |
| Zinc gluconate dihydrate 50% granules | 150.38 |
| Diluents | |
| Mannitol Powder USP/NF | 45.00 |
| Mannitol Granules USP/NF | 360.09 |
| Microcrystalline Cellulose NF/Ph. Eur. | 198.00 |
| Disintegrants | |
| Crospovidone USP | 63.00 |
| Sod. Starch Glycolate NF | 18.00 |
| Emulsifiers | |
| Polysorbate 80 NF | 2.25 |
| Sodium Lauryl Sulfate NF | 0.90 |
| Sorbitan Monostearate | 4.50 |
| Binder | |
| Povidone USP/NF | 1.80 |
| Hardness Enhancer | |
| Polyethylene Glycol 8000NF | 9.00 |
| Lubricants | |
| Magnesium Stearate NF | 7.20 |
| Purified talc USP | 3.60 |
| Silicon dioxide NF | 1.80 |
| Flavor | |
| Black Cherry Natural Sweet Type- | 11.70 |
| Flavor 207J98 | |
| Sweetener | |
| Sucralose NF | 4.50 |
| Acesulfame K FCC | 2.25 |
| Color | |
| FD&C Red No. 40 Aluminum Lake HT | 0.90 |
| Total | 900.00 |

### Example 10

### a) Formula for Zinc Carbonate 50% Granules (Polymer: Drug Ratio is 1:4)

| Material Name | mg/dose |
|---|---|
| Active | |
| Zinc Carbonate USP | 19.18 |
| (19.18 × 52.15/100 = 10.00 mg) | |
| Bioadhesive | |
| Hydroxy propyl methyl cellulose | 4.80 |
| K100 LV Premium USP/EP | |
| Diluents | |
| Microcrystalline cellulose NF/Ph. Eur. | 14.39 |
| Solvent | |
| Purified water USP | q.s. |
| Total | 38.36 |

### b) Formula for Zinc Rapid Melt 14.5 mg Long Acting Tablets—Black Cherry Flavor

| Material Name | mg/tablet |
|---|---|
| Active | |
| Milled Zinc Acetate Dihydrate | 15.12 |
| (15.12 * 29.78/100 = 4.50 mg) | |
| Zinc Carbonate 50% granules | 38.36 |
| Diluents | |
| Mannitol Powder USP/NF | 45.00 |
| Mannitol Granules USP/NF | 472.14 |
| Microcrystalline Cellulose NF/Ph. Eur. | 198.00 |
| Disintegrants | |
| Crospovidone USP | 63.00 |
| Sod. Starch Glycolate NF | 18.00 |
| Emulsifiers | |
| Polysorbate 80 NF | 2.25 |
| Sodium Lauryl Sulfate NF | 0.90 |
| Sorbitan Monostearate | 4.50 |
| Binder | |
| Povidone USP/NF | 1.80 |
| Hardness Enhancer | |
| Polyethylene Glycol 8000NF | 9.00 |
| Lubricants | |
| Magnesium Stearate NF | 7.20 |
| Purified talc USP | 3.60 |
| Silicon dioxide NF | 1.80 |
| Flavor | |
| Black Cherry Natural Sweet Type- | 11.70 |
| Flavor 207J98 | |
| Sweetener | |
| Sucralose NF | 4.50 |
| Acesulfame K FCC | 2.25 |
| Color | |
| FD&C Red No. 40 Aluminum Lake HT | 0.90 |
| Total | 900.00 |

### Example 11

### a) Formula for Zinc Carbonate 50% Granules (Polymer: Drug Ratio is 1:4)

| Material Name | mg/dose |
|---|---|
| Active | |
| Zinc Carbonate USP | 19.18 |
| (19.18 × 52.15/100 = 10.00 mg) | |
| Bioadhesive | |
| Hydroxy propyl methyl cellulose | 4.80 |
| K100 M CR USP/EP | |
| Diluents | |
| Microcrystalline cellulose NF/Ph. Eur. | 14.39 |
| Solvent | |
| Purified water USP | q.s. |
| Total | 38.36 |

### b) Formula for Zinc Rapid Melt 14.5 mg Long Acting Tablets—Black Cherry Flavor

| Material Name | mg/tablet |
|---|---|
| Active | |
| Milled Zinc Acetate Dihydrate | 15.12 |
| (15.12 * 29.78/100 = 4.50 mg) | |
| Zinc Carbonate 50% granules | 38.36 |
| Diluents | |
| Mannitol Powder USP/NF | 45.00 |
| Mannitol Granules USP/NF | 472.14 |
| Microcrystalline Cellulose NF/Ph. Eur. | 198.00 |
| Disintegrants | |
| Crospovidone USP | 63.00 |
| Sod. Starch Glycolate NF | 18.00 |
| Emulsifiers | |
| Polysorbate 80 NF | 2.25 |
| Sodium Lauryl Sulfate NF | 0.90 |
| Sorbitan Monostearate | 4.50 |
| Binder | |
| Povidone USP/NF | 1.80 |
| Hardness Enhancer | |
| Polyethylene Glycol 8000NF | 9.00 |
| Lubricants | |
| Magnesium Stearate NF | 7.20 |
| Purified talc USP | 3.60 |
| Silicon dioxide NF | 1.80 |
| Flavor | |
| Black Cherry Natural Sweet Type- | 11.70 |
| Flavor 207J98 | |
| Sweetener | |
| Sucralose NF | 4.50 |
| Acesulfame K FCC | 2.25 |
| Color | |
| FD&C Red No. 40 Aluminum Lake HT | 0.90 |
| Total | 900.00 |

### Example 12

a) Formula for Zinc Carbonate 50% Granules (Polymer: Drug Ratio is 1:4)

| Material Name | mg/dose |
|---|---|
| Active | |
| Zinc Carbonate USP | 19.18 |
| (19.18 × 52.15/100 = 10.00 mg) | |
| Bioadhesive | |
| Hydroxy propyl methyl cellulose | 4.80 |
| K 4M CR USP/EP | |
| Diluents | |
| Microcrystalline cellulose NF/Ph. Eur. | 14.39 |
| Solvent | |
| Purified water USP | q.s. |
| Total | 38.36 |

### b) Formula for Zinc Rapid Melt 14.5 mg Long Acting Tablets—Black Cherry Flavor

| Material Name | mg/tablet |
|---|---|
| Active | |
| Milled Zinc Acetate Dihydrate | 15.12 |
| (15.12 * 29.78/100 = 4.50 mg) | |
| Zinc Carbonate 50% granules | 38.36 |
| Diluents | |
| Mannitol Powder USP/NF | 45.00 |
| Mannitol Granules USP/NF | 472.14 |
| Microcrystalline Cellulose NF/Ph. Eur. | 198.00 |
| Disintegrants | |
| Crospovidone USP | 63.00 |
| Sod. Starch Glycolate NF | 18.00 |
| Emulsifiers | |
| Polysorbate 80 NF | 2.25 |
| Sodium Lauryl Sulfate NF | 0.90 |
| Sorbitan Monostearate | 4.50 |
| Binder | |
| Povidone USP/NF | 1.80 |
| Hardness Enhancer | |
| Polyethylene Glycol 8000NF | 9.00 |
| Lubricants | |
| Magnesium Stearate NF | 7.20 |
| Purified talc USP | 3.60 |
| Silicon dioxide NF | 1.80 |
| Flavor | |
| Black Cherry Natural Sweet Type- | 11.70 |
| Flavor 207J98 | |
| Sweetener | |
| Sucralose NF | 4.50 |
| Acesulfame K FCC | 2.25 |
| Color | |
| FD&C Red No. 40 Aluminum Lake HT | 0.90 |
| Total | 900.00 |

### Example 13

### a) Formula for Zinc Carbonate 50% Granules (Polymer: Drug Ratio is 1:4)

| Material Name | mg/dose |
|---|---|
| Active | |
| Zinc Carbonate USP | 19.18 |
| (19.18 × 52.15/100 = 10.00 mg) | |
| Bioadhesive | |
| Hydroxy propyl methyl cellulose | 4.80 |
| K15 M CR USP/EP | |
| Diluents | |
| Microcrystalline cellulose NF/Ph. Eur. | 14.39 |
| Solvent | |
| Purified water USP | q.s. |
| Total | 38.36 |

### b) Formula for Zinc Rapid Melt 14.5 mg Long Acting Tablets—Black Cherry Flavor

| Material Name | mg/tablet |
|---|---|
| Active | |
| Milled Zinc Acetate Dihydrate | 15.12 |
| (15.12 * 29.78/100 = 4.50 mg) | |
| Zinc Carbonate 50% granules | 38.36 |
| Diluents | |
| Mannitol Powder USP/NF | 45.00 |
| Mannitol Granules USP/NF | 472.14 |
| Microcrystalline Cellulose NF/Ph. Eur. | 198.00 |
| Disintegrants | |
| Crospovidone USP | 63.00 |
| Sod. Starch Glycolate NF | 18.00 |
| Emulsifiers | |
| Polysorbate 80 NF | 2.25 |
| Sodium Lauryl Sulfate NF | 0.90 |
| Sorbitan Monostearate | 4.50 |
| Binder | |
| Povidone USP/NF | 1.80 |
| Hardness Enhancer | |
| Polyethylene Glycol 8000NF | 9.00 |
| Lubricants | |
| Magnesium Stearate NF | 7.20 |
| Purified talc USP | 3.60 |
| Silicon dioxide NF | 1.80 |
| Flavor | |
| Black Cherry Natural Sweet Type- | 11.70 |
| Flavor 207J98 | |
| Sweetener | |
| Sucralose NF | 4.50 |
| Acesulfame K FCC | 2.25 |
| Color | |
| FD&C Red No. 40 Aluminum Lake HT | 0.90 |
| Total | 900.00 |

### Example 14

### a) Formula for Zinc Carbonate 50% Granules (Polymer: Drug Ratio is 1:4)

| Material Name | mg/dose |
|---|---|
| Active | |
| Zinc Carbonate USP | 19.18 |
| (19.18 × 52.15/100 = 10.00 mg) | |
| Bioadhesive | |
| Polycarbophil USP | 4.80 |
| Diluents | |
| Microcrystalline cellulose NF/Ph. Eur. | 14.39 |
| Solvent | |
| Purified water USP | q.s. |
| Total | 38.36 |

### b) Formula for Zinc Rapid Melt 14.5 mg Long Acting Tablets—Black Cherry Flavor

| Material Name | mg/tablet |
|---|---|
| Active | |
| Milled Zinc Acetate Dihydrate | 15.12 |
| (15.12 * 29.78/100 = 4.50 mg) | |
| Zinc Carbonate 50% granules | 38.36 |
| Diluents | |
| Mannitol Powder USP/NF | 45.00 |
| Mannitol Granules USP/NF | 472.14 |
| Microcrystalline Cellulose NF/Ph. Eur. | 198.00 |
| Disintegrants | |
| Crospovidone USP | 63.00 |
| Sod. Starch Glycolate NF | 18.00 |
| Emulsifiers | |
| Polysorbate 80 NF | 2.25 |
| Sodium Lauryl Sulfate NF | 0.90 |
| Sorbitan Monostearate | 4.50 |
| Binder | |
| Povidone USP/NF | 1.80 |
| Hardness Enhancer | |
| Polyethylene Glycol 8000NF | 9.00 |
| Lubricants | |
| Magnesium Stearate NF | 7.20 |
| Purified talc USP | 3.60 |
| Silicon dioxide NF | 1.80 |
| Flavor | |
| Black Cherry Natural Sweet Type- | 11.70 |
| Flavor 207J98 | |
| Sweetener | |
| Sucralose NF | 4.50 |
| Acesulfame K FCC | 2.25 |
| Color | |
| FD&C Red No. 40 Aluminum Lake HT | 0.90 |
| Total | 900.00 |

### Preparation of Zinc Salt 50% Granules:

Sift Emcocel 90M, Zinc salt and polymers through #30 mesh. Above ingredients are dry mixed for about 10 min.
Dry mixed material is granulated by adding purified water to get desired wet granules
Dry the wet mass in Tray dryer at 50° C.±5° C. until the moisture content is in between 1.0-1.5%. Sift Dried granules through #40 Screen.
Single Layer tablet Procedure:
Part 1: Mix ¾ part of Pearlitol 200SD, vivapur 102 (micro crystalline cellulose) and Pearlitol 160C then pass through #30 mesh.
Part 2: Mix #40 mesh pass Zinc acetate and zinc gluconate granules and add to part 1 mixture.
Part 3: Sift remaining ¼ part of pearlitol 200 SD, Explotab, Sorbitan Monostearate and SLS passes through #30 mesh
Part 4: Tween-80K is adsorbed on Polyplasdone XL-10 in a Petri dish. Then pass through #40 screen.
Part 5: Black cherry flavor, Sucralose, Acesulfame pass through #40 mesh.
Mix Part 1, Part 3, Part 4, Part 5 in a double cone blender. Blend for 10 min. Magnesium Stearate, Talc and Sylloid pass through #50 mesh and add to the Blending part and Blend for 5 min using double cone blender.
Compress the blend part into tablets using tablet compression machine

What is claimed is:

1. A pharmaceutical composition for the controlled release of an active within the oral cavity containing a pharmaceutical active salt micro-encapsulated within a carrier selected from the group consisting of bioadhesive/muco-adhesive polymers, biopolymers, fats, waxes, gums and mixtures thereof which is further incorporated within a fast dissolving polymer matrix selected from the group consisting of cellulose and cellulose derivatives, thermoplastic polymers, hydrogels, gums and mixtures thereof.

2. The pharmaceutical composition of claim 1 wherein said active is a therapeutically effective amount of a pharmaceutical active selected from the group consisting of anti-bacterial agents, anti-tussives, expectorants, anti-viral agents, anti-inflammatory agents, neurotherapeutic agents, analgesics, anti-fungal agents, anti-neoplastic agents, anti-histamines, proteins, enzymes, minerals, hormonal agents, non-steroidal anti-inflammatories, cytokines, steroids, nicotine, insulin, anti-tussives, antihistamines, decongestants, alkaloids, mineral supplements, laxatives, vitamins, antacids, ion exchange resins, anti-cholesterolemics, anti-arrhythmics, anti-pyretics, analgesics, appetite suppressants, expectorants, anti-anxiety agents, anti-ulcer agents, anti-inflammatory substances, coronary dilators, cerebral dilators, peripheral vasodilators, anti-infectives, psycho-tropics, anti-maniocs, stimulants, gastrointestinal agents, sedatives, anti-diarrheal preparations, anti-angina drugs, vasodilators, anti-hypertensive drugs, vasoconstrictors, migraine treatments, antibiotics, tranquilizers, anti-psychotics, antitumor drugs, anticoagulants, antithrombotic drugs, hypnotics, anti-emetics, anti-nauseates, anti-consultants, neuromuscular drugs, hyper- and hypo-glycolic, spasmodic, uterine relaxants, mineral and nutritional additives, anti-obesity drugs, anabolic drugs, erythropoietin drugs, anti-asthmatics, cough suppressants, mucolytics, anti-uricemic drugs and mixtures thereof.

3. The pharmaceutical composition of claim 2 formulated as a rapid melt tablet wherein said pharmaceutical active salt is selected from the group consisting of organic and inorganic mineral salts.

4. The pharmaceutical composition of claim 3 formulated as a rapid melt tablet wherein said pharmaceutical active salt is selected from the group consisting of zinc, copper, and other organic and inorganic mineral salts.

5. The composition of claim 4 wherein said organic and inorganic salts are selected from the group consisting of zinc bromide, zinc chloride, zinc iodine, zinc fluoride, zinc ammonium sulfate, zinc chromate, zinc fluorosilicate, zinc dithionate, zinc sulfate, zinc nitrate, zinc phosphate, zinc fluorozirconate, zinc oxide, and mixtures thereof.

6. The composition of claim 5 wherein said organic salts of zinc are selected from the group consisting of zinc citrate, zinc acetate, zinc gluconate, zinc asparate, zinc ascorbate, zinc oroate, zinc divalent amino acid zinc salts, zinc succinate, zinc tartrate, zinc glycero-phosphate, zinc salicylate, zinc formate, and mixtures thereof.

7. The composition of claim 6 wherein said organic salts of zinc are zinc acetate and zinc gluconate.

8. The composition of claim 7 wherein said bioadhesive/muco-adhesive polymer is selected from the group comprising cellulose derivatives methylcellulose, ethyl cellulose, hydroxy-ethylcellulose, hydroxyl-propyl cellulose, hydroxy propyl methylcellulose, sodium carboxy methylcellulose, poly(acrylic acid) polymers (carbomers, polycarbophil), poly(hydroxyethyl methylacrylate), oly (ethylene oxide), poly-vinyl pyrrolidone, poly (vinyl alcohol), natural polymers, tragacanth, sodium alginate, karaya gum, guar gum, xanthan gum, lecithin, soluble starch, gelatin, pectin, chitosan. and mixtures thereof.

9. The composition of claim 8 wherein said thermoplastic polymers include the erodible neutral polystyrene and semi-crystalline bio-erodible polymers, selected from the group consisting of poly-anhydrides and polylactic acid. polyvinyl alcohol, polyamides, polycarbonates, polyalkylene glycols, polyvinyl ethers, esters and halides, polymethacrylic acid, polymethylmethacrylic acid, methyl cellulose, hydroxylpropyl cellulose, hydroxylpropyl methyl cellulose, and sodium carboxy methyl cellulose and mixtures thereof.

10. The composition of claim 9 wherein said tablet is a bi-layered round lozenge shaped tablet, comprised of white colored layer and an orange colored layer containing zinc acetate, zinc gluconate and vitamin C along with zinc acetate and zinc gluconate incorporated in the tablets in amounts of from about 5.0 to about 25.0 mgs.

11. The composition of claim 10 wherein said zinc acetate and zinc gluconate are incorporated in the tablets in amounts of from about 10.0 to about 15.0 mgs.

12. The composition of claim 11 wherein the composition is a flash-bead, capsulated microspheres or a compressed tablet.

13. The composition of claim 12 further comprising a diluent/bulking material selected from the group consisting of mannitol, dextrate, sorbitol, glycerol and mixtures thereof.

14. The composition of claim 13 further comprising a salivating agent.

15. The composition of claim 14, wherein the salivating agent is an emulsifier.

16. The composition of claim 15, wherein the emulsifier is sodium lauryl sulfate.

17. The composition of claim 16 wherein the emulsifier is polysorbate 80.

18. The composition of claim 17 further comprising a binder

19. The composition of claim 18 wherein the binder is polyethylene glycol.

20. The composition of claim 19, wherein the diluent/bulking material further comprises magnesium stearate.

21. The composition of claim 20, further comprising a sweetener and flavor agents.

22. A rapid-melt compressed solid pharmaceutical composition comprising crosspovidone, mannitol, stearic acid, and a mineral salt.
